(19) United States
(12) Patent Application Publication
(10) Pub. No.: US 2007/0073101 A1
(43) Pub. Date: Mar. 29, 2007

(54) Fiberscope with a separable insertion tube

(76) Inventors: Alain Queyroux, Gueret (FR); Francois Jouineau, Gueret (FR); Pierre Faugeras, Limoges (FR)

Correspondence Address: Ralph A. Dowell of DOWELL & DOWELL P.C., 2111 Eisenhower Ave, Suite 406, Alexandria, VA, 22314, US

(21) Appl. No.: 10/573,963

(22) PCT Filed: Sep. 29, 2004
(86) PCT No.: PCT/FR04/02465

§ 371 Date: Mar. 30, 2006

(30) Foreign Application Priority Data
Sep. 30, 2003 (FR) 0311438

(51) Int. Cl. A61B 1/00

(52) U.S. Cl. 600/102

(57) ABSTRACT

The inventive fiberscope comprises a body (10), an insertion tube belonging to a part (20) which is detachable with respect to said body (10), a first (40) and a second (50) mechanical connection and optical transmission elements which are removably jointed in service. Each element (40, 50) is connected to corresponding guiding means (42, 52) in such a way that a motion produced by guiding means (42) of the body (10) is transmitted to guiding means (52) of the detachable part (20). Said elements (40, 50) are also used for transmitting light from optical means (60) of the body (10) towards optical means (70, 72) of the detachable part (20) and for transmitting an image from optical means (70, 72) of the detachable part (20) to optical means (60) of the body (10).

The present invention relates to a fiberscope with a separable insertion tube.

A fiberscope is a flexible endoscope permitting exploration of the deep cavities of the body. To this end, this fiberscope comprises a body, and an insertion tube that is intended to penetrate into the aforementioned cavities.

In recent times, the effectiveness of medical endoscopy procedures has increased by virtue of technological advances that have, among other things, permitted production of endoscopes of ever smaller size and, consequently, endoscopes that cause less trauma to the patient.

In addition, these endoscopy examinations, which are being performed in increasing numbers, have become indispensable in routine practice. They permit, on the one hand, rapid and reliable diagnosis and, on the other hand, regular monitoring of many pathological conditions, in most cases meaning it is not necessary to resort to other types of examinations that are more costly or more aggressive.

Given that they necessitate the use of sophisticated optical and electro-optical equipment, endoscopes generally entail high costs so that, for reasons of economy, they have to be reused on a large number of patients. Under these conditions, reuse necessitates sterilization or at least disinfection of the endoscope between two successive patients.

However, such sterilization does not always prove completely reliable, which leads to risks of contamination of patients. The reason for this is that endoscopes do not withstand very high temperatures, with the result that this sterilization is carried out using procedures whose reliability cannot be regarded as absolute, in particular with regard to emergent microorganisms, such as that implicated in Creutzfeldt-Jakob disease.

Moreover, such sterilization takes a relatively long time and entails the involvement of specific personnel and the acquisition of premises and equipment for protecting the individual. Moreover, this sterilization entails the use of toxic substances whose efficacy is limited in time and which may possibly be harmful to the environment.

In conclusion, such endoscopes prove expensive, such that the routine examinations using them are particularly cost-intensive.

To overcome these disadvantages, EP-A-0 813 384 has proposed an endoscope whose insertion tube can be separated from the body. Thus, after each use, this tube is detached and discarded and is replaced by a new insertion tube.

This makes it possible to dispense with a sterilization operation. This is because the soiled tube is no longer reused, while the body of the endoscope, which is situated outside the patient, does not need to be sterilized after each examination.

This being the case, the object of the invention is to make available a fiberscope with a separable insertion tube, having a simple mechanical structure and a relatively low manufacturing cost, while permitting rapid and convenient connection between the body and the insertion tube.

To this end, the invention relates to a fiberscope comprising a body and an insertion tube belonging to a part that is separable from the body, this body and this separable part being mechanically joined at a connection zone, this fiberscope also comprising:
first guide means belonging to the body, in particular a first set of cables that can be operated via a maneuvering element belonging to the body, in particular a handle;
second guide means belonging to the separable part, in particular a second set of cables that are able to move the insertion tube;
first optical means belonging to the body and able to transmit light to the connection zone and return an image of this connection zone to a zone for viewing by a practitioner, such as an eyepiece;
second optical means belonging to the separable part and able to transmit light from the connection zone to a distal end of the insertion tube and return an image from this distal end of the insertion tube to the connection zone and
first and second mechanical connection and optical transmission elements, which are joined removably in service, each element being integral with corresponding guide means in such a way that a movement imparted by the first guide means can be transmitted to the second guide means, these mechanical connection and optical transmission elements also being able to transmit light coming from the first optical means to the second optical means and to return an image from the second optical means to the first optical means.

According to other characteristics of the invention:
The connection zone defines a seat having inner walls in the shape of a portion of a sphere, while at least one of the first and second mechanical connection and optical transmission elements has spherical outer walls with a diameter substantially equal to that of said inner walls, so as to allow three degrees of freedom in rotation, without any degree of freedom in translation, of these two elements relative to the walls of the seat.
The first and second mechanical connection and optical transmission elements are joined removably, in service, by being mutually fixed in a removable manner.
A first mechanical connection and optical transmission element, provided with said outer spherical walls, defines a groove for receiving in a removable manner a second mechanical connection and optical transmission element, which is in particular a plane disk.
The first mechanical connection and optical transmission element has two parallel front faces and a protruding crown defining, with one of these front faces, said receiving groove.
The connection zone comprises two complementary, almost semicylindrical connection portions belonging respectively to the body and to the separable part, in which connection portions corresponding recesses are formed which are intended to form said seat in service.
The first and second mechanical connection and optical transmission elements are joined removably in service by being wedged relative to one another, in particular by being pressed flat against one another.
The first optical means comprise a succession of lenses associated with a light source.
The second optical means comprise a central bundle of optical fibers that are able to return an image from the distal end of the insertion tube to the connection zone, and also a peripheral bundle of optical fibers that are able to transmit light from the connection zone to this distal end.
The peripheral bundle is surrounded by a sheath, in particular made of a metal or plastic material.
The central bundle is made up of separate optical fibers.
The central bundle is formed by different individual bundles of optical fibers of polyhedral shape which are disposed side by side one another.
The connection zone is surrounded by an external locking means, in particular a ring.

The invention will be better understood, and other advantages of the invention will become clearer, from the following description of a fiberscope according to the principle of the invention, which description is given solely by way of non-limiting example and with reference to the attached drawings, in which:

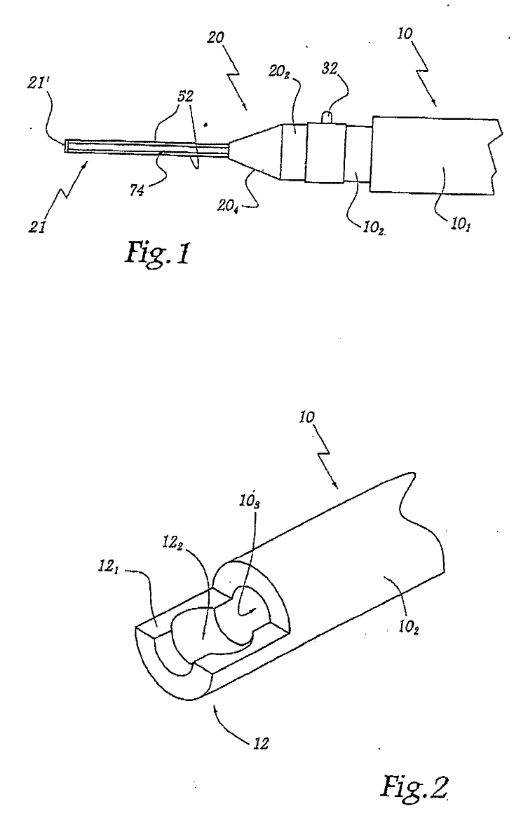

FIG. 1 is a front view schematically depicting a fiberscope according to the invention;

FIG. 2 is a perspective view depicting the connection zone between the body and the insertion tube of the fiberscope from FIG. 1;

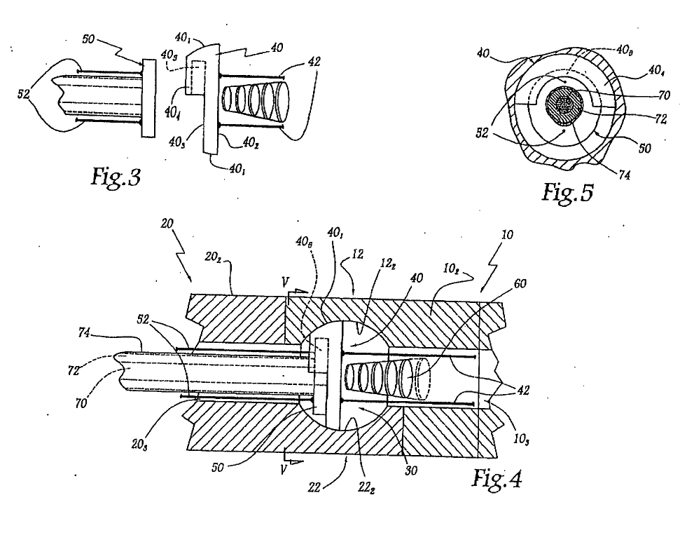

FIG. 3 is a side view depicting mechanical connection and optical transmission elements included in the fiberscope in FIGS. 1 and 2;

FIG. 4 is a view in longitudinal section depicting the aforementioned connection zone in which the mechanical connection and optical transmission elements from FIG. 3 are disposed; and

FIG. 5 is a sectional view along the line V-V in FIG. 4.

As is shown particularly in FIG. 1, the fiberscope of the invention comprises a body 10 intended to be gripped by a user. This body is provided with a handle (not shown) with which it is possible in particular to maneuver guide cables, which will be described in more detail below.

The body 10 comprises a sleeve 10, continued by a coaxial shaft 102 of smaller diameter. As is shown in particular in FIGS. 2 and 5, a central bore 10₃ is formed inside this sleeve 10₁ and this shaft 10₂.

The fiberscope also has an end part 20 which is separable from the body 10 and which comprises a cylindrical shaft 20₂ extending in the continuation of the shaft 10₂ of the body 10. This shaft 20₂ is itself continued by a connecting region 20₄ of truncated cone shape, which is terminated by an insertion tube 21 intended to penetrate into deep cavities in the patient. As is shown in FIG. 5, the shaft 20₂ has a central bore 20₃ running through it and coaxial to the one 10₃ formed in the body.

This body 10 and this separable part 20 are connected mechanically, at a connection zone which is formed by two connection portions 12 and 22, visible in FIG. 5, belonging respectively to the body 10 and to the separable part 20. In FIG. 2, only the connection portion 12 is shown, it being understood that the structure of the portion 22 is analogous.

As is shown in this FIG. 2, the connection portion 12 has overall a semicylindrical shape, thereby forming a partial continuation of the shaft 10₂. This portion 12 is truncated by a diametral flat surface 12₁, starting from which a recess is hollowed out whose walls 12₂ have the shape of a portion of a sphere. Similarly, the portion 22 is truncated by a flat surface, starting from which there extends a recess whose walls 22₂ also have the shape of a portion of a sphere.

Therefore, in service, the two portions 12 and 22 are in mutual contact via their respective flat surfaces, of which only one 12₁ is illustrated. Moreover, the two recesses mentioned above form a seat 30 having spherical walls 12₂ and 22₂ and in which the bores 10₃ and 20₃ open out. This connection zone 12, 22 is surrounded by a supplementary locking means, which in this case is a ring 32.

FIG. 3 depicts mechanical connection and optical transmission elements with which the fiberscope according to the invention is equipped. A transparent disk 40 is thus provided forming a lens, the outer wall 40₁ of which forms a sphere segment, with a diameter corresponding to that of the walls 12₂ and 22₂ of the seat 30.

This disk 40 is truncated by a first front face 40₂ on which is fixed a first set of cables 42. These, which are for example at least two in number, are fixed on the disk 40 by any suitable means.

The second front face 40₃ of the disk 40, parallel to the face 40₂ mentioned above, defines, with an end crown 40₄, a groove 40₅. The latter is intended to receive a transparent flat disk 50 of overall circular shape. This disk 50 receives a second set of cables 52, which are at least two in number, and which are fixed by any suitable means.

In service, as is shown in FIG. 5, the disk 50 is inserted into the inner volume of the groove 405 delimited by the disk 40. In this way, these two disks 40 and 50 are mutually joined, both in translation and in rotation.

Consequently, a displacement of the cables 42, initiated by the handle, is transmitted in a corresponding manner to the cables 52 by way of these two disks 40 and 50. The cables 52 extend as far as the distal end of the insertion tube 21, which end is formed by a flat lens 21′ (FIG. 1), where they are fixed by any suitable means.

It will be appreciated therefore that the aforementioned moving of the cables 42, which induces a corresponding movement of the cables 52, causes a displacement of the insertion tube 21 in the form of a twisting. With a view to facilitating such a movement, the outer wall of this tube 21, which is formed by a sheath, as will be seen below, is advantageously configured in the manner of a bellows.

The fiberscope according to the invention is provided, in the area of the body 10, with a conventional optical system with which it is possible, on the one hand, to send light in the direction of the disk 40 and, on the other hand, to return an image from the latter. Such a system, which is formed for example by a succession of lenses associated with a light source, is represented schematically in FIG. 4, where it is designated by reference number 60. Alternatively, this optical system can also comprise an arrangement of optical fibers.

Similarly, the separable part 20 is provided with means which, in the first instance, make it possible to transmit, to the distal end of the insertion tube 21, the light coming from the optical system 60, via the disks 40 and 50. These means also make it possible to return the image, emitted at this distal end of the tube 21, to the disks 40 and 50 and, consequently, to the optical system 60, in such a way as to permit viewing by the practitioner.

More specifically, such optical means comprise two bundles of fibers, namely in the first instance a cylindrical central bundle 70 able to return the image from the distal end of the insertion tube 21. This bundle 70 is surrounded by a peripheral annular bundle 72 intended to send light toward this distal end.

Advantageously, in a configuration not shown here, the central bundle 70 is formed by different individual bundles of fibers of polyhedral shape which are disposed side by side one another.

As is shown in particular in FIGS. 4 and 5, the peripheral bundle 72 is surrounded by a protective sheath 74, which is made of metal or plastic for example. The bundles 70 and 72, associated with the sheath 74, continue beyond the shaft 20₂ of the separable part 20, in such a way as to form the insertion tube 21, as is shown in FIG. 1. Beyond this shaft, as is also shown in said FIG. 1, these bundles 66 and 72 and this sheath 74 are surrounded by the cables 52, which are fixed on the lens 21′, as has been mentioned above.

After use of the fiberscope according to the invention, it is first necessary to remove the ring 32, then to disconnect the separable part 20 from the body 10 by disengaging the disk 50 from the groove 40₅. This separable part, soiled in the area of the insertion tube 21, is then discarded and replaced by another separable part of similar structure, with a view to supplementary use of the fiberscope according to the invention.

The invention is not limited to the example that has been described and shown.

Thus, the mechanical connection and optical transmission disks 40 and 50 may be formed in other configurations. Moreover, operating channels may adjoin the different optical fibers for the purpose of aspiration, coagulation, or for passage of biopsy forceps or other operating instruments.

In service, the two elements 40 and 50 can be joined in a removable manner by being fixed removably as in the illustrated example. Alternatively, they can also be wedged relative to one another, within their seat, in particular by being pressed flat against one another, in such a way that they are mutually joined both in terms of rotation and translation, when in service.

In a further alternative, at least one of the mechanical connection and optical-transmission elements can be provided with a pivot. This measure makes it possible to force the element in question to enter into rotation about a single axis.

In a further alternative, the central bundle 70, making it possible to return the image from the distal end of the insertion tube to the connection zone, can be formed by separate optical fibers.

The invention has its application in all fields of human or animal endoscopy. In the medical field, it will be used in particular in ear, nose and throat endoscopy, in endoscopy of the esophagus, stomach, duodenum and colon, in bronchoscopy, in urology, or in gynecology.

The objectives mentioned above are able to be achieved with the invention.

Thus, the insertion tube, belonging to the fiberscope of the invention, can be set in motion in a reliable manner by virtue of the transmission of the movements imparted via the handle of the body of the fiberscope. Such mechanical transmission of the movements can be employed in all directions, which allows precise guiding of the insertion tube.

Moreover, the presence of the disks 40 and 50, which provide a supplementary function of optical transmission, guarantees the transmission of light from the body to the end of the tube, and that of the images from this end to the body.

In addition, the mechanical connection present between the body and the separable part of the fiberscope is extremely simple and quick to use. This consequently provides for considerable ease of use and allows disposable sterile insertion tubes to be employed. In this regard, these insertion tubes can be received, after sterilization by x-ray, in packages which are themselves sterile, thus guaranteeing rigorously aseptic conditions.

It will also be noted that the invention introduces a concept of traceability of the different insertion tubes. Thus, each tube can be assigned a specific reference.

Once used, the insertion tube of the fiberscope according to the invention can be disposed of in an environmentally acceptable manner, for example by incineration. This makes it possible to avoid using toxic substances, in particular proteolytic agents, which are not reprocessed and which are released into the environment.

Finally, the invention makes it possible to dispense with the customary decontamination procedures. In this way, it affords a significant reduction in labor and by and large obviates the dangerous tasks associated with decontamination prevalent in the prior art. The invention also permits a substantial reduction in the costs associated with examinations using fiberscopes.

1. A fiberscope comprising a body (10) and an insertion tube (21) belonging to a part (20) that is separable from the body (10), this body (10) and this separable part (20) being mechanically joined at a connection zone (12, 22), this fiberscope also comprising:
first guide means belonging to the body (10), in particular a first set of cables (42), that can be operated via a maneuvering element belonging to the body, in particular a handle;
second guide means belonging to the separable part (20), in particular a second set of cables (52), that are able to move the insertion tube (21);
first optical means (60) belonging to the body (10), and able to transmit light to the connection zone (12, 22) and return an image of this connection zone (12, 22) to a zone for viewing by a practitioner, such as an eyepiece;
second optical means (70, 72) belonging to the separable part (20) and able to transmit light from the connection zone (12, 22) to a distal end (21′) of the insertion tube (21) and return an image from this distal end of the insertion tube (21) to the connection zone (12, 22), and
first (40) and second (50) mechanical connection and optical transmission elements which are joined removably in service, each element (40, 50) being integral with corresponding guide means (42, 52) in such a way that a movement imparted by the first guide means (42) can be transmitted to the second guide means (52), these mechanical connection and optical transmission elements (40, 50) also being able to transmit light coming from the first optical means (60) to the second optical means (70, 72) and to return an image from the second optical means (70, 72) to the first optical means (60).

2. The fiberscope according to claim 1, characterized in that the connection zone (12, 22) defines a seat (30) having inner walls (12₂, 22₂) in the shape of a portion of a sphere, while at least one of the first (40) and second (50) mechanical connection and optical transmission elements has spherical outer walls (40₁) with a diameter substantially equal to that of said inner walls, so as to allow three degrees of freedom in rotation, without any degree of freedom in translation, of these two elements relative to the walls of the seat (30).

3. The fiberscope according to claim 1, characterized in that the first (40) and second (50) mechanical connection and optical transmission elements are joined removably, in service, by being mutually fixed in a removable manner.

4. The fiberscope according to claim 3, characterized in that a first (40) mechanical connection and optical transmission element, provided with said outer spherical walls (40₁), defines a groove (40₅) for receiving, in a removable manner, a second (50) mechanical connection and optical transmission element, which is in particular a plane disk.

5. The fiberscope according to claim 4, characterized in that the first (40) mechanical connection and optical transmission element has two parallel front faces (40₂, 40₃) and a protruding crown (40₄) defining, with one (40₃) of these front faces, said receiving groove (40₅).

6. The fiberscope according to claim 2, characterized in that the connection zone comprises two complementary, almost semicylindrical connection portions (12, 22) belonging respectively to the body (10) and to the separable part (20), in which connection portions corresponding recesses are formed which are intended to form said seat (30) in service.

7. The fiberscope according to claim 1, characterized in that the first and second mechanical connection and optical transmission elements are joined removably in service by being wedged relative to one another, in particular by being pressed flat against one another.

8. The fiberscope according to claim 1, characterized in that the first optical means (60) comprise a succession of lenses associated with a light source.

9. The fiberscope according to claim 1, characterized in that the second optical means comprise a central bundle (70) of optical fibers that are able to return an image from the distal end of the insertion tube (21) to the connection zone (12, 22), and also a peripheral bundle (72) of optical fibers that are able to transmit light from the connection zone (12, 22) to this distal end.

10. The fiberscope according to claim 9, characterized in that the peripheral bundle (72) is surrounded by a sheath (74), in particular made of a metal or plastic material.

11. The fiberscope according to claim 9, characterized in that the central bundle (70) is made up of separate optical fibers.

12. The fiberscope according to claim 9, characterized in that the central bundle is formed by different individual bundles of optical fibers of polyhedral shape which are disposed side by side one another.

13. The fiberscope according to claim 1, characterized in that the connection zone (12, 22) is surrounded by an external locking means, in particular a ring (32).
